(19) United States
(12) Patent Application Publication
(10) Pub. No.: US 2009/0280133 A1
(43) Pub. Date: Nov. 12, 2009

(54) PHARMACEUTICAL COMPOUNDS AS INHIBITORS OF CELL PROLIFERATION AND THE USE THEREOF

(75) Inventors: Kazuyuki Suzuki, Salt Lake City, UT (US); Tamara Artz, Salt Lake City, UT (US); Warren S. Weiner, Salt Lake City, UT (US); Yevgeniya Klimova, Sandy, UT (US)

Correspondence Address: Myriad PHARMACEUTICALS, Inc.;c/o CPA Global, P.O. Box 52050, Minneapolis, MN, 55402, US

(73) Assignee: Myriad Genetics, Incorporated, Salt Lake City, UT (US)

(21) Appl. No.: 12/465,786

(22) Filed: May 14, 2009

Related U.S. Application Data
(63) Continuation of application No. PCT/US07/84613, filed on Nov. 14, 2007.
(60) Provisional application No. 60/865,758, filed on Nov. 14, 2006.

(51) Int. Cl. A61K 39/395; C07D 239/42; A61K 31/505; A61P 35/00

(52) U.S. Cl. 424/178.1; 544/326; 514/256; 544/329; 514/274; 544/317

(57) ABSTRACT

Disclosed are compounds of Formula I effective as cytotoxic agents. The compounds of this invention are useful in the treatment of a variety of clinical conditions in which uncontrolled growth and spread of abnormal cells occurs.

### CROSS REFERENCE TO RELATED APPLICATIONS

This application is a continuation of PCT/US07/84613, filed on Nov. 14, 2007, which claims the benefit of U.S. Provisional Application Ser. No. 60/865,758, filed on Nov. 14, 2006, both of which are incorporated herein by reference in their entirety.

### FIELD OF THE INVENTION

This invention is in the field of medicinal chemistry. In particular, the invention relates to compounds that are cytotoxic agents. The invention also relates to the use of these compounds as therapeutically effective anti-cancer agents.

### BACKGROUND OF THE INVENTION

Cancer is a common cause of death in the world; about 10 million new cases occur each year, and cancer is responsible for 12% of deaths worldwide, making cancer the third leading cause of death. World Health Organization, National Cancer Control Programmes: Policies and Managerial Guidelines (2d ed. 2002)

Despite advances in the field of cancer treatment, the leading therapies to date include surgery, radiation, and chemotherapy. Chemotherapeutic approaches are said to fight cancers that are metastasized or that are particularly aggressive. Most of the cancer chemotherapy agents currently in clinical use are cytotoxins. Cytotoxic agents work by damaging or killing cells that exhibit rapid growth. Ideal cytotoxic agents would have specificity for cancer and tumor cells, while not affecting normal cells. Unfortunately, none have been found and instead agents that target especially rapidly dividing cells (both tumor and normal) have been used.

Accordingly, discovery of new and effective treatments for cancer is a high priority for health care researchers. Materials that are cytotoxic to cancer cells while exerting only mild effects on normal cells are highly desirable. For this reason, there remains a definite need in the art for new effective chemotherapeutic agents.

### BRIEF SUMMARY OF THE INVENTION

The present invention is related to the discovery that compounds of Formula I below, are cytotoxic agents. Thus, they are useful in treating or delaying the onset of diseases and disorders that are responsive to cytotoxic agents.

Accordingly, one aspect of the present invention is directed to the use of compounds of the present invention in treating or ameliorating neoplasm and cancer, by administering the compounds to cells in vitro or in vivo in warm-blooded animals, particularly mammals.

Many of the compounds as represented by Formula I below are novel compounds. Therefore, another aspect of the present invention is to provide novel compounds.

Yet another aspect of the present invention is to provide a pharmaceutical composition useful for treating disorders responsive to cytotoxic agents, containing an effective amount of a compound of the present invention, preferably in admixture with one or more pharmaceutically acceptable carriers or diluents.

In yet another aspect of the present invention, methods are provided for the preparation of the novel compounds of the present invention.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION OF THE INVENTION

It has been discovered that compounds of the present invention are potent and highly efficacious cytotoxic agents. Therefore, the compounds are useful for treating diseases and disorders responsive to cytotoxic agents.

The above various methods of the present invention can be practiced by or comprise treating cells in vitro or a warm-blooded animal, particularly mammal, more particularly a human with an effective amount of a compound according to the present invention. As used herein, the phrase “treating . . . with . . . a compound” means either administering the compound to cells or an animal, or administering to cells or an animal the compound or another agent to cause the presence or formation of the compound inside the cells or the animal. Preferably, the methods of the present invention comprise administering to cells in vitro or to a warm-blooded animal, particularly mammal, more particularly a human a pharmaceutical composition comprising an effective amount of a compound according to the present invention.

Specifically, the methods of the present invention comprise treating cells in vitro or a warm-blooded animal, particularly mammal, more particularly a human with an effective amount of a compound according to Formula I:

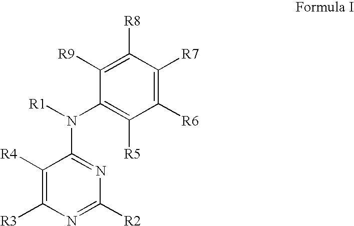

or a pharmaceutically acceptable salt or solvate thereof, wherein:
R₁ is methyl;
R₂ is C₁₋₆ alkyl, C₂₋₆ alkenyl, C₂₋₆ alkynyl, C₁₋₆ alkoxy, halo, C₁₋₆ haloalkyl, C₁₋₆ haloalkoxy, C₁₋₆ alkylthio, amino, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S(═O)₂NH₂, —S(═O)₂N(C₁₋₆ alkyl)₂, —S(═O)₂NH(C₁₋₆ alkyl), —S(═O)₂aryl, —S(═O)₂heteroaryl, —CN, —N₃, and —NO₂, each optionally substituted with one or more substituents;
R₃ and R₄ are independently H, C₁₋₆ alkyl, C₁₋₆ carbocyclic, C₁₋₆ alkylamino, C₂₋₆ alkenyl, C₂₋₆ alkynyl, C₁₋₆ alkoxy, halo, C₁₋₆ haloalkyl, C₁₋₆ haloalkoxy, C₁₋₆ alkylthio, amino, heterocyclic, aryl, heteroaryl, —N-aryl, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S(═O)₂NH₂, —S(═O)₂N(C₁₋₆ alkyl)₂, —S(═O)₂NH(C₁₋₆ alkyl), —S-aryl, —CN, —N₃, —NH₂ and —NO₂, each optionally substituted with one or more substituents;
R₅ and R₉ are independently H or F;
R₆ and R₈ are independently H, C₁₋₆ alkyl, C₁₋₆ carbocyclic, C₁₋₆ alkylamino, C₂₋₆ alkenyl, C₂₋₆ alkynyl, C₁₋₆ alkoxy, halo, C₁₋₆ haloalkyl, C₁₋₆ haloalkoxy, C₁₋₆ alkylthio, amino, heterocyclic, aryl, heteroaryl, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —N-aryl, —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S(═O)₂NH₂, —S(═O)₂N(C₁₋₆ alkyl)₂, —S(═O)₂NH(C₁₋₆ alkyl), —S-aryl, —S(═O)₂aryl, —S(═O)₂heteroaryl, —CN, —N₃, and —NO₂, each optionally substituted with one or more substituents;
R₇ is C₁₋₆ alkyl, C₁₋₆ carbocyclic, C₂₋₆ alkenyl, C₂₋₆ alkynyl, C₁₋₆ alkoxy, halo, halo-C₁₋₆ alkyl, halo-C₁₋₆ alkoxy, C₁₋₆ alkylthio, amino, heterocyclic, aryl, heteroaryl, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —N-aryl, —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S-aryl, —S(═O)₂NH₂, —S(═O)₂N(C₁₋₆ alkyl)₂, —S(═O)₂NH(C₁₋₆ alkyl), —S(═O)₂-aryl, N—S(═O)₂NH₂—CN, —N₃, —NH₂ and —NO₂, each optionally substituted with one or more substituents;
with the provisos that:
1) when R₂ is NH₂ or NHCH₃, then R₃ is not methyl or substituted phenyl, R₄ is not NH₂ or halo;
2) when R₂ is halo, then R₃ is not methyl, R₄ is not halo, R₇ is not NH-acetate or N(Me)(Acetate);
3) when R₂ is methyl then R₃ is not N-aryl or R₄ is not NO₂; and
4) when R₂ is OH then R₃ is not OH.

In some embodiments R₂ is C₁₋₄ alkyl, C₂₋₄ alkenyl, C₂₋₄ alkynyl, C₁₋₄ alkoxy, halo, C₁₋₄ haloalkyl, C₁₋₄ haloalkoxy, C₁₋₄ alkylthio, amino, —NH(C₁₋₄ alkoxy), —NH(C₁₋₄ alkyl)OH, —NHS(═O)₂(C₁₋₄ alkyl), —NHS(═O)₂(aryl), —NH(C₁₋₄ alkyl)C(═O)OH, —NH(C₁₋₄ alkyl)C(═O)O(C₁₋₄ alkyl), —C(═O)OH, —C(═O)O(C₁₋₄ alkyl), —C(═O)NH₂, —C(═O)NH(C₁₋₄ alkyl), —C(═O)N(C₁₋₄ alkyl)₂, —OH, —O-aryl, —SH, C₁₋₄ haloalkylthio, —S(═O)₂NH₂, —S(═O)₂N(C₁₋₄ alkyl)₂, —S(═O)₂NH(C₁₋₄ alkyl), —CN, —N₃, and —NO₂, each optionally substituted with one or more substituents. In certain embodiments R₂ is methyl, CN, aminomethyl, Cl, SCH₃, NH₂, NHCH₃, NHCH₂CH₂OH, N(CH₃)₂, NH—OCH₃, or NHCH₂COOH.

In some embodiments R₃ and R₄ are independently H, C₁₋₄ alkyl, C₁₋₄ carbocyclic, amino, C₁₋₄ alkoxy, halo, C₁₋₄ haloalkyl, C₁₋₄ haloalkoxy, heterocyclic, aryl, heteroaryl, —NHS(═O)₂(C₁₋₄ alkyl), —NHS(═O)₂(aryl), —NH(C₁₋₄ alkyl)C(═O)OH, —NH(C₁₋₄ alkyl)C(═O)O(C₁₋₄ alkyl), —C(═O)OH, —C(═O)O(C₁₋₄ alkyl), —C(═O)NH₂, —C(═O)NH(C₁₋₄ alkyl), —C(═O)N(C₁₋₄ alkyl)₂, —OH, —O-aryl, C₁₋₄ haloalkylthio, —S(═O)₂NH₂, —S(═O)₂N(C₁₋₄ alkyl)₂, —S(═O)₂NH(C₁₋₄ alkyl), —CN, —N₃, and —NO₂, each optionally substituted with one or more substituents.

In some embodiments R₅, R₆, R₈ and R₉ are H or F.

In some embodiments R₇ is C₁₋₄ alkyl, C₁₋₄ carbocyclic, C₂₋₄ alkenyl, C₂₋₄ alkynyl, C₁₋₄ alkoxy, halo, C₁₋₄ haloalkyl, C₁₋₄ haloalkoxy, C₁₋₄ alkylthio, amino, heterocyclic, aryl, heteroaryl, —NH(C₁₋₄ alkoxy), —NH(C₁₋₄ alkyl)OH, —NHS(═O)₂(C₁₋₄ alkyl), —NHS(═O)₂(aryl), —NH(C₁₋₄ alkyl)C(═O)OH, —NH(C₁₋₄ alkyl)C(═O)O(C₁₋₄ alkyl), —C(═O)OH, —C(═O)O(C₁₋₄ alkyl), —C(═O)NH₂, —C(═O)NH(C₁₋₄ alkyl), —C(═O)N(C₁₋₄ alkyl)₂, —OH, —O-aryl, —SH, C₁₋₄ haloalkylthio, —S(═O)₂NH₂, —S(═O)₂N(C₁₋₄ alkyl)₂, —S(═O)₂NH(C₁₋₄ alkyl), —CN, —N₃, —NH₂ and —NO₂, each optionally substituted with one or more substituents. In certain embodiments R₇ is C₁₋₄ alkyl, —OH, C₁₋₄ alkoxy, —SH, C₁₋₄ alkylthio, —S(═O)₂NH₂, —S(═O)₂N(C₁₋₄ alkyl)₂, —S(═O)₂NH(C₁₋₄ alkyl), each optionally substituted with one or more substituents.

In certain embodiments compounds of the invention include compounds of Formula I or pharmaceutically acceptable salts or solvates thereof, wherein:

R₁ is methyl;
R₂ is methyl, CN, aminomethyl, Cl, SCH₃, NH₂, NHCH₃, NHCH₂CH₂OH, N(CH₃)₂, NH—OCH₃, or NHCH₂COOH;
R₃ and R₄ are independently H, CH₃, COOH, COOCH₃, COOCH₂CH₃, phenyl, Cl or NHS(═O)₂(Ph-4-OCH₃);

### R₅, R₆, R₈ and R₉ are H;

### R₇ is OH or OCH₃;

with the provisos that:

1) when R₂ is NH₂ or NHCH₃, then R₃ is not methyl, R₄ is not NH₂ or halo;

2) when R₂ is halo, then R₃ is not methyl, R₄ is not halo; and

3) when R₂ is methyl then R₄ is not NO₂.

Among all the compounds of the present invention as disclosed above, preferred are those that are cytotoxic as determined by the method and under conditions described in Example 14, preferably at an EC₅₀ of no greater than about 1,000 nM, more preferably at an EC₅₀ of no greater than about 500 nM, more preferably at an EC₅₀ of no greater than about 200 nM, even more preferably at an EC₅₀ of no greater than about 100 nM, and most preferably at an EC₅₀ of no greater than about 10 nM.

Exemplary compounds of the present invention are compounds provided in Examples 1-12, and pharmaceutically acceptable salts or prodrugs thereof. Specific exemplary compounds include but are not limited to: (2-Chloro-pyrimidin-4-yl)-(4-methoxyphenyl)-methyl amine;
Methyl 2-chloro-6-[(4-methoxyphenyl)(methyl)amino]pyrimidine-4-carboxylate;
Ethyl 4-[(4-methoxyphenyl)(methyl)amino]-2-(methylthio)pyrimidine-5-carboxylate;
N⁴-(4-Methoxyphenyl)-N²,N²,N⁴,6-tetramethylpyrimidine-2,4-diamine;
N⁴-(4-Methoxyphenyl)-N²,N²,N⁴-trimethylpyrimidine-2,4-diamine;
4-[(4-Methoxyphenyl)(methyl)amino]pyrimidine-2-carbonitrile;
4-[(4-Methoxyphenyl)(methyl)amino]-6-methylpyrimidine-2-carbonitrile;
6-Chloro-N⁴-(4-methoxyphenyl)-N⁴-methylpyrimidine-2,4-diamine;
2-(Aminomethyl)-N-(4-methoxyphenyl)-N-methylpyrimidin-4-amine;
4-Methoxyphenyl)-methyl-(2-methyl-6-phenyl-pyrimidin-4-yl)amine;
(2,6-dimethyl-pyrimidin-4-yl)-(4-methoxyphenyl)-methylamine;
4-Methoxy-N-{4-[(4-methoxyphenyl)-methylamino]-2-methyl-pyrimidin-5-yl)-benzenesulfonamide; and
N-{4-[(4-hydroxyphenyl)-methylamino]-2-methyl-pyrimidin-5-yl)-4-methyoxy-benzenesulfonamide.

Unless specifically stated otherwise or indicated by a bond symbol (dash or double dash), the connecting point to a recited group will be on the right-most stated group. Thus, for example, a hydroxyalkyl group is connected to the main structure through the alkyl and the hydroxyl is a substituent on the alkyl.

The term “alkyl” as employed herein by itself or as part of another group refers to both straight and branched chain radicals of up to ten carbons. Useful alkyl groups include straight-chained and branched C₁₋₁₀ alkyl groups, more preferably C₁₋₆ alkyl groups. Typical C₁₋₁₀ alkyl groups include methyl, ethyl, propyl, isopropyl, butyl, sec-butyl, tert-butyl, 3-pentyl, hexyl and octyl groups, which may be optionally substituted.

The term “alkenyl” as employed herein by itself or as part of another group means a straight or branched chain radical of 2-10 carbon atoms, unless the chain length is limited thereto, including at least one double bond between two of the carbon atoms in the chain. Typical alkenyl groups include ethenyl, 1-propenyl, 2-propenyl, 2-methyl-1-propenyl, 1-butenyl and 2-butenyl.

The term “alkynyl” is used herein to mean a straight or branched chain radical of 2-10 carbon atoms, unless the chain length is limited thereto, wherein there is at least one triple bond between two of the carbon atoms in the chain. Typical alkynyl groups include ethynyl, 1-propynyl, 1-methyl-2-propynyl, 2-propynyl, 1-butynyl and 2-butynyl.

Useful alkoxy groups include oxygen substituted by one of the C₁₋₁₀ alkyl groups mentioned above, which may be optionally substituted.

Useful alkylthio groups include sulfur substituted by one of the C₁₋₁₀ alkyl groups mentioned above, which may be optionally substituted. Also included are the sulfoxides and sulfones of such alkylthio groups.

Useful amino groups include —NH₂, —NHRₓ, and —NRₓR_{y}, wherein Rₓ and R_{y} are C₁₋₁₀ alkyl or cycloalkyl groups. The alkyl group may be optionally substituted.

Optional substituents include one or more substituents chosen from hydroxyl, halo, alkyl, alkenyl, alkynyl, alkoxy, haloalkyl, haloalkoxy, amino, —C(═O)OH, —C(═O)O(C₁₋₃ alkyl), C₁₋₆ alkyl-C(═O)O(C₁₋₃ alkyl), C₁₋₆ alkyl-C(═O)OH, C₁₋₆ alkyl-C(═O)NH(C₁₋₃ alkyl), C₁₋₆ alkyl-C(═O)N(C₁₋₃ alkyl)₂, —C(═O)NH₂, —C(═O)NH(C₁₋₃ alkyl), —C(═O)N(C₁₋₃ alkyl)₂, —S(═O)₂(C₁₋₃alkyl), —S(═O)₂NH₂, —S(═O)₂N(C₁₋₃ alkyl)₂, —S(═O)₂NH(C₁₋₃ alkyl), —SH, —SCF₃, —CN, —NH₂, and —NO₂

The term “aryl” as employed herein by itself or as part of another group refers to monocyclic, bicyclic or tricyclic aromatic groups containing from 6 to 14 carbons in the ring portion.

Useful aryl groups include C₆₋₁₄ aryl, preferably C₆₋₁₀ aryl. Typical C₆₋₁₄ aryl groups include phenyl, naphthyl, phenanthrenyl, anthracenyl, indenyl, azulenyl, biphenyl, biphenylenyl and fluorenyl groups.

The term “carbocycle” as employed herein include cycloalkyl and partially saturated carbocyclic groups. Useful cycloalkyl groups are C₃₋₈ cycloalkyl. Typical cycloalkyl groups include cyclopropyl, cyclobutyl, cyclopentyl, cyclohexyl and cycloheptyl.

Useful saturated or partially saturated carbocyclic groups are cycloalkyl groups as described above, as well as cycloalkenyl groups, such as cyclopentenyl, cycloheptenyl and cyclooctenyl.

Useful halo or halogen groups include fluorine, chlorine, bromine and iodine.

The term “arylalkyl” is used herein to mean any of the above-mentioned C₁₋₁₀ alkyl groups substituted by any of the above-mentioned C₆₋₁₄ aryl groups. Preferably the arylalkyl group is benzyl, phenethyl or naphthylmethyl.

The term “arylalkenyl” is used herein to mean any of the above-mentioned C₂₋₁₀ alkenyl groups substituted by any of the above-mentioned C₆₋₁₄ aryl groups.

The term “arylalkynyl” is used herein to mean any of the above-mentioned C₂₋₁₀ alkynyl groups substituted by any of the above-mentioned C₆₋₁₄ aryl groups.

The term “aryloxy” is used herein to mean oxygen substituted by one of the above-mentioned C₆₋₁₄ aryl groups, which may be optionally substituted. Useful aryloxy groups include phenoxy and 4-methylphenoxy.

The term “arylalkoxy” is used herein to mean any of the above mentioned C₁₋₁₀ alkoxy groups substituted by any of the above-mentioned aryl groups, which may be optionally substituted. Useful arylalkoxy groups include benzyloxy and phenethyloxy.

The term “haloalkyl” is used herein to mean C₁₋₁₀ alkyl groups substituted by one or more fluorine, chlorine, bromine or iodine atoms, e.g., fluoromethyl, difluoromethyl, trifluoromethyl, pentafluoroethyl, 1,1-difluoroethyl, chloromethyl, chlorofluoromethyl and trichloromethyl groups.

Useful acylamino (acylamido) groups are any C₁₋₆ acyl(alkanoyl) attached to an amino nitrogen, e.g., acetamido, chloroacetamido, propionamido, butanoylamido, pentanoylamido and hexanoylamido, as well as aryl-substituted C₁₋₆ acylamino groups, e.g., benzoylamido, and pentafluorobenzoylamido.

Useful acyloxy groups are any C₁₋₆ acyl(alkanoyl) attached to an oxy (—O—) group, e.g., formyloxy, acetoxy, propionyloxy, butanoyloxy, pentanoyloxy and hexanoyloxy.

The term heterocycle is used herein to mean a saturated or partially saturated 3-7 membered monocyclic, or 7-10 membered bicyclic ring system, which consists of carbon atoms and from one to four heteroatoms independently selected from the group consisting of O, N, and S, wherein the nitrogen and sulfur heteroatoms can be optionally oxidized, the nitrogen can be optionally quaternized, and including any bicyclic group in which any of the above-defined heterocyclic rings is fused to a benzene ring, and wherein the heterocyclic ring can be substituted on a carbon or on a nitrogen atom if the resulting compound is stable, including an oxo substituent (“═O”) wherein two hydrogen atoms are replaced.

Useful saturated or partially saturated heterocyclic groups include tetrahydrofuranyl, pyranyl, piperidinyl, piperazinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups. The term “heteroaryl” as employed herein refers to groups having 5 to 14 ring atoms; 6, 10 or 14 π electrons shared in a cyclic array; and containing carbon atoms and 1, 2 or 3 oxygen, nitrogen or sulfur heteroatoms. Useful heteroaryl groups include thienyl(thiophenyl), benzo[b]thienyl, naphtho[2,3-b]thienyl, thianthrenyl, furyl(furanyl), isobenzofuranyl, chromenyl, xanthenyl, phenoxathiinyl, pyrrolyl, including without limitation 2H-pyrrolyl, imidazolyl, pyrazolyl, pyridyl(pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzisoxazol-3-yl, benzimidazolyl, 2-oxindolyl and 2-oxobenzimidazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

The term “heteroaryloxy” is used herein to mean oxygen substituted by one of the above-mentioned heteroaryl groups, which may be optionally substituted. Useful heteroaryloxy groups include pyridyloxy, pyrazinyloxy, pyrrolyloxy, pyrazolyloxy, imidazolyloxy and thiophenyloxy.

The term “heteroarylalkoxy” is used herein to mean any of the above-mentioned C₁₋₁₀ alkoxy groups substituted by any of the above-mentioned heteroaryl groups, which may be optionally substituted.

Some of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

Examples of pharmaceutically acceptable addition salts include inorganic and organic acid addition salts, and inorganic and organic base addition salts with bases.

Examples of prodrugs of the compounds of the invention include the simple esters of carboxylic acid containing compounds; esters of hydroxy containing compounds; imines of amino containing compounds; carbamate of amino containing compounds; and acetals and ketals of alcohol containing compounds.

The compounds of this invention may be prepared using methods known to those skilled in the art, or the novel methods of this invention. Specifically, the compounds of this invention with Formula I can be prepared as illustrated by the exemplary reactions in Schemes 1-2.

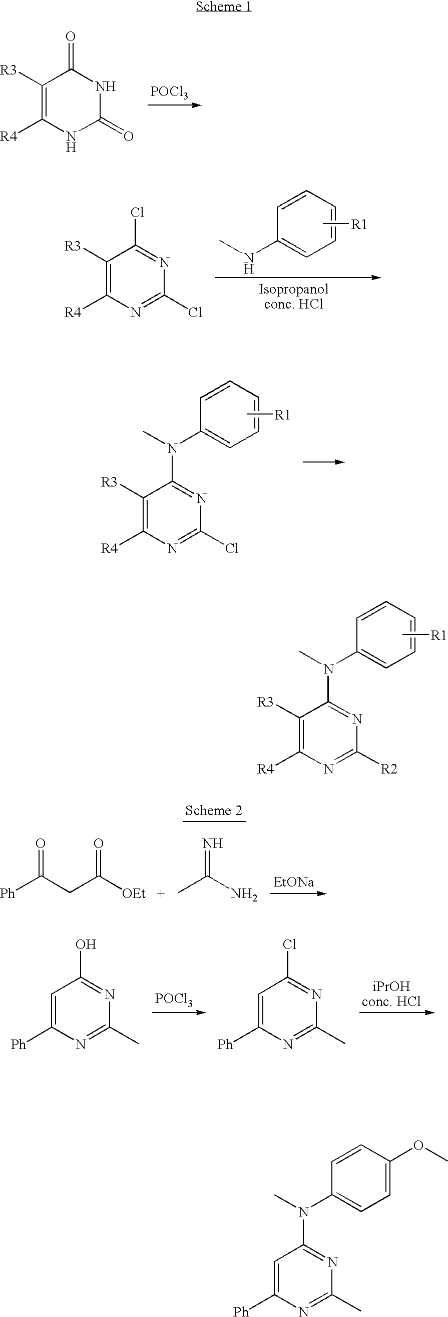

An important aspect of the present invention is the discovery that compounds having Formula I are cytotoxic agents. Therefore, these compounds are useful in treating diseases that are responsive to cytotoxic agents. For example, these compounds are useful in a variety of clinical conditions in which there is uncontrolled cell growth and spread of abnormal cells, such as in the case of cancer.

The present invention also includes a therapeutic method comprising administering to an animal an effective amount of a compound, or a pharmaceutically acceptable salt or prodrug of said compound of Formula I, wherein said therapeutic method is useful to treat cancer, which is a group of diseases characterized by the uncontrolled growth and spread of abnormal cells.

In practicing the therapeutic methods, effective amounts of compositions containing therapeutically effective concentrations of the compounds formulated for oral, intravenous, local and topical application, for the treatment of neoplastic diseases and other diseases, are administered to an individual exhibiting the symptoms of one or more of these disorders. The amounts are effective to ameliorate or eliminate one or more symptoms of the disorders. An effective amount of a compound for treating a particular disease is an amount that is sufficient to ameliorate, or in some manner reduce, the symptoms associated with the disease. Such amount may be administered as a single dosage or may be administered according to a regimen, whereby it is effective. The amount may cure the disease but, typically, is administered in order to ameliorate the symptoms of the disease. Typically, repeated administration is required to achieve the desired amelioration of symptoms.

Another aspect of the present invention is to provide a pharmaceutical composition, containing an effective amount of a compound of Formula I, or a pharmaceutically acceptable salt or solvate of said compound, in admixture with one or more pharmaceutically acceptable carriers or diluents.

In one embodiment, a pharmaceutical composition comprising a compound of Formula I disclosed herein, or a pharmaceutically acceptable salt or solvate of said compound, in combination with a pharmaceutically acceptable vehicle is provided.

Preferred pharmaceutical compositions comprise compounds of Formula I, and pharmaceutically acceptable salts, esters, or prodrugs thereof, that are cytotoxic as determined by the method described in Example 14, preferably at an EC₅₀ no greater than 1,000 nM, more preferably at an EC₅₀ no greater than 500 nM, more preferably at an EC₅₀ no greater than 200 nM, more preferably at an EC₅₀ no greater than 100, and most preferably at an EC₅₀ no greater than 10 nM.

Another embodiment of the present invention is directed to a composition effective to inhibit neoplasia comprising a compound, or a pharmaceutically acceptable salt or prodrug of said compound of Formula I, which functions as a cytotoxic agent, in combination with at least one known cancer chemotherapeutic agent, or a pharmaceutically acceptable salt or solvate of said agent. Examples of known cancer chemotherapeutic agents which may be used for combination therapy include, but not are limited to alkylating agents, antimitotic agents, topo I inhibitors, topo II inhibitors, RNA/DNA antimetabolites, DNA antimetabolites, EGFR inhibitors, proteosome inhibitors, and antibodies.

In practicing the methods of the present invention, the compound of the invention may be administered together with at least one known chemotherapeutic agent as part of a unitary pharmaceutical composition. Alternatively, the compound of the invention may be administered apart from at least one known cancer chemotherapeutic agent. In one embodiment, the compound of the invention and at least one known cancer chemotherapeutic agent are administered substantially simultaneously, i.e. the compounds are administered at the same time or one after the other, so long as the compounds reach therapeutic levels in the blood at the same time. On another embodiment, the compound of the invention and at least one known cancer chemotherapeutic agent are administered according to their individual dose schedule, so long as the compounds reach therapeutic levels in the blood.

It has been reported that alpha-1-adrenoceptor antagonists can inhibit the growth of prostate cancer cell via induction of apoptosis (Kyprianou, N., et al., Cancer Res 60:4550-4555, (2000)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known alpha-1-adrenoceptor antagonists, or a pharmaceutically acceptable salt or solvate of said agent.

It has been reported that sigma-2 receptors are expressed in high densities in a variety of tumor cell types (Vilner, B. J., et al., Cancer Res. 55: 408-413 (1995)) and that sigma-2 receptor agonists activate a novel apoptotic pathway and potentiate antineoplastic drugs in breast tumor cell lines. (Kyprianou, N., et al., Cancer Res. 62:313-322 (2002)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known sigma-2 receptor agonist, or a pharmaceutically acceptable salt or solvate of said agonist.

It has been reported that combination therapy with a HMG-CoA reductase inhibitor, and butyrate, an inducer of apoptosis in the Lewis lung carcinoma model in mice, showed potentiating antitumor effects (Giermasz, A., et al., Int. J. Cancer 97:746-750 (2002)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known HMG-CoA reductase inhibitor, or a pharmaceutically acceptable salt or solvate of said agent.

It has been reported that HIV protease inhibitors have potent anti-angiogenic activities and promote regression of Kaposi sarcoma (Sgadari, C., et al., Nat. Med. 8:225-232 (2002)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known HIV protease inhibitor, or a pharmaceutically acceptable salt or solvate of said agent.

It has been reported that synthetic retinoids have good activity in combination with other chemotherapeutic agents in small-cell lung cancer cell lines (Kalemkerian, G. P., et al., Cancer Chemother. Pharmacol. 43:145-150 (1999)). Synthetic retinoids have also been reported to have good activity in combination with gamma-radiation on bladder cancer cell lines (Zou, C., et al., Int. J. Oncol. 13:1037-1041 (1998)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known retinoid and synthetic retinoid, or a pharmaceutically acceptable salt or solvate of said agent.

It has been reported that proteasome inhibitors exert anti-tumor activity in vivo and in tumor cells in vitro, including those resistant to conventional chemotherapeutic agents. By inhibiting NF-kappaB transcriptional activity, proteasome inhibitors may also prevent angiogenesis and metastasis in vivo and further increase the sensitivity of cancer cells to apoptosis (Almond, J. B., et al., Leukemia 16:433-443 (2002)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known proteasome inhibitor, or a pharmaceutically acceptable salt or solvate of said agent.

It has been reported that tyrosine kinase inhibitors have potent synergetic effect in combination with other anti-leukemic agents (Liu, W. M., et al. Br. J. Cancer 86:1472-1478 (2002)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known tyrosine kinase inhibitor, or a pharmaceutically acceptable salt or solvate of said agent.

It has been reported that prenyl-protein transferase inhibitors possess preclinical antitumor activity against human breast cancer (Kelland, L. R., et. al., Clin. Cancer Res. 7:3544-3550 (2001)). Synergy of a protein farnesyltransferase inhibitor and cisplatin in human cancer cell lines also has been reported (Adjei, A. A., et al., Clin. Cancer. Res. 7:1438-1445 (2001)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known prenyl-protein transferase inhibitor, including farnesyl protein transferase inhibitor, inhibitors of geranylgeranyl-protein transferase type I (GGPTase-I) and geranylgeranyl-protein transferase type-II, or a pharmaceutically acceptable salt or solvate of said agent.

It has been reported that cyclin-dependent kinase (CDK) inhibitors have potent synergetic effect in combination with other anticancer agents, such as a DNA topoisomerase I inhibitor in human colon cancer cells (Motwani, M., et al., Clin. Cancer Res. 7:4209-4219, (2001)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known cyclin-dependent kinase inhibitor, or a pharmaceutically acceptable salt or solvate of said agent.

It has been reported that in preclinical studies COX-2 inhibitors were found to block angiogenesis, suppress solid tumor metastases, and slow the growth of implanted gastrointestinal cancer cells (Blanke, C. D., Oncology (Huntingt) 16(No. 4 Suppl. 3):17-21 (2002)). Therefore, another embodiment of the present invention is directed to a composition effective to inhibit neoplasia comprising a compound, or a pharmaceutically acceptable salt, solvate, or prodrug of a compound described herein, which functions as a cytotoxic agent, in combination with at least one known COX-2 inhibitor, or a pharmaceutically acceptable salt or solvate of said inhibitor.

Another embodiment of the present invention is directed to a composition effective to inhibit neoplasia comprising a bioconjugate of a compound described herein, which functions as a cytotoxic agent, in bioconjugation with at least one known therapeutically useful antibody, growth factors, cytokines, or any molecule that binds to the cell surface. The antibodies and other molecules will deliver a compound described herein to its targets and make it an effective anticancer agent. The bioconjugates could also enhance the anticancer effect of therapeutically useful antibodies.

Similarly, another embodiment of the present invention is directed to a composition effective to inhibit neoplasia comprising a compound, or a pharmaceutically acceptable salt or prodrug of a compound described herein, which functions as a cytotoxic agent, in combination with radiation therapy. In this embodiment, the compound of the invention may be administered at the same time as the radiation therapy is administered or at a different time.

Yet another embodiment of the present invention is directed to a composition effective for post-surgical treatment of cancer, comprising a compound, or a pharmaceutically acceptable salt or prodrug of a compound described herein, which functions as a cytotoxic agent. The invention also relates to a method of treating cancer by surgically removing the cancer and then treating the animal with one of the pharmaceutical compositions described herein.

Stent implantation has become the new standard angioplasty procedure. However, in-stent restenosis remains the major limitation of coronary stenting. New approaches have been developed to target pharmacological modulation of local vascular biology by local administration of drugs. This allows for drug applications at the precise site and time of vessel injury. Numerous pharmacological agents with antiproliferative properties are currently under clinical investigation, including actinomycin D, rapamycin or paclitaxel coated stents (Regar E., et al., Br. Med. Bull. 59:227-248 (2001)). Therefore, apoptosis inducers, which are antiproliferative, are useful as therapeutics for the prevention or reduction of in-stent restenosis.

Another important aspect of the present invention is the surprising discovery that compounds of the present invention are potent and highly efficacious cytotoxic agents even in drug resistant cancer cells, which enables these compounds to inhibit the growth and proliferation of drug resistant cancer cells, and to cause cell death in the drug resistant cancer cells. Specifically, the compounds of the present invention are not substrates for the MDR transporters such as Pgp-1 (MDR-1), MRP-1 and BCRP. This is particularly surprising in view of the fact that many commercially available chemotherapeutics are substrates for multidrug resistance transporters (MDRs).

Multidrug resistance is the major cause of chemotherapy failure. Drug resistance is typically caused by ATP-dependent efflux of drug from cells by ATP-binding cassette (ABC) transporters. In particular, the ABC transporters ABCB1 (MDR-1, P glycoprotein); ABCC1 (MRP1); and ABCG2 (BCRP, MXR) are typically over-expressed in drug resistant tumors and thus are implicated in drug resistance. In comparison to most standard anti-cancer drugs, which are not effective in killing drug resistant cancer cells, the compounds of the present invention are effective in killing drug resistant cancer cells. Therefore, compounds of this invention are useful for the treatment of drug resistant cancer.

Thus, another aspect of the present invention is the application of the methods and compounds of the present invention as described above to tumors that have acquired resistance to other anticancer drugs. In one embodiment, a compound of the present invention is administered to a cancer patient who has been treated with another anti-cancer drug. In another embodiment, a compound of the present invention is administered to a patient who has been treated with and is not responsive to another anti-cancer drug or developed resistance to such other anti-cancer compound. In another embodiment, a compound of the present invention is administered to a patient who has been treated with another anti-cancer drug and is refractory to said other anti-cancer drug. The compounds of the present invention can be used in treating cancer in a patient who is not responsive or is resistant to any other anti-cancer agent. Examples of such other anti-cancer agent may include alkylating agents, antimitotic agents, topo I inhibitors, topo II inhibitors, RNA/DNA antimetabolites, EGFR inhibitors, angiogenesis inhibitors, tubulin inhibitors, proteosome inhibitors, etc.

Pharmaceutical compositions within the scope of this invention include all compositions wherein the compounds of the present invention are contained in an amount that is effective to achieve its intended purpose. While individual needs vary, determination of optimal ranges of effective amounts of each component is within the skill of the art. Typically, the compounds may be administered to animals, e.g., mammals, orally at a dose of 0.0025 to 50 mg/kg of body weight, per day, or an equivalent amount of the pharmaceutically acceptable salt thereof, to a mammal being treated. Preferably, approximately 0.01 to approximately 10 mg/kg of body weight is orally administered. For intramuscular injection, the dose is generally approximately one-half of the oral dose. For example, a suitable intramuscular dose would be approximately 0.0025 to approximately 25 mg/kg of body weight, and most preferably, from approximately 0.01 to approximately 5 mg/kg of body weight. If a known cancer chemotherapeutic agent is also administered, it is administered in an amount that is effective to achieve its intended purpose. The amounts of such known cancer chemotherapeutic agents effective for cancer are well known to those skilled in the art.

The unit oral dose may comprise from approximately 0.01 to approximately 50 mg, preferably approximately 0.1 to approximately 10 mg of the compound of the invention. The unit dose may be administered one or more times daily, as one or more tablets, each containing from approximately 0.1 to approximately 10 mg, conveniently approximately 0.25 to 50 mg of the compound or its solvates.

In a topical formulation, the compound may be present at a concentration of approximately 0.01 to 100 mg per gram of carrier.

In addition to administering the compound as a raw chemical, the compounds of the invention may be administered as part of a pharmaceutical preparation containing suitable pharmaceutically acceptable carriers comprising excipients and auxiliaries, which facilitate processing of the compounds into preparations that may be used pharmaceutically. Preferably, the preparations, particularly those preparations which may be administered orally and that may be used for the preferred type of administration, such as tablets, dragees, and capsules, and also preparations that may be administered rectally, such as suppositories, as well as suitable solutions for administration by injection or orally, contain from approximately 0.01 to 99 percent, preferably from approximately 0.25 to 75 percent of active compound(s), together with the excipient.

Also included within the scope of the present invention are the non-toxic pharmaceutically acceptable salts of the compounds of the present invention. Acid addition salts are formed by mixing a solution of the compounds of the present invention with a solution of a pharmaceutically acceptable non-toxic acid. Basic salts are formed by mixing a solution of the compounds of the present invention with a solution of a pharmaceutically acceptable non-toxic base.

The pharmaceutical compositions of the invention may be administered to any animal, which may experience the beneficial effects of the compounds of the invention. Foremost among such animals are mammals, e.g., humans and veterinary animals, although the invention is not intended to be so limited.

The pharmaceutical compositions of the present invention may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner, which is itself known, e.g., by means of conventional mixing, granulating, dragee-making, dissolving, or lyophilizing processes. Thus, pharmaceutical preparations for oral use may be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular: fillers, cellulose preparations and/or calcium phosphates, as well as binders. If desired, disintegrating agents may be added, such as starches and also carboxymethyl-starch, cross-linked polyvinyl pyrrolidone, agar, or alginic acid or a salt thereof. Auxiliaries are, above all, flow-regulating agents and lubricants. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations are used. Dye stuffs or pigments may be added to the tablets or dragee coatings, e.g., for identification or in order to characterize combinations of active compound doses.

Other pharmaceutical preparations, which may be used orally include push-fit capsules made of gelatin, as well as soft, sealed capsules made of gelatin and a plasticizer. The push-fit capsules may contain the active compounds in the form of: granules, which may be mixed with fillers, binders, and/or lubricants, and, optionally, stabilizers. In soft capsules, the active compounds are preferably dissolved or suspended in suitable liquids, such as fatty oils, or liquid paraffin. In addition, stabilizers may be added.

Possible pharmaceutical preparations, which may be used rectally include, e.g., suppositories, which consist of a combination of one or more of the active compounds with a suppository base. Suitable suppository bases are, e.g., natural or synthetic triglycerides, or paraffin hydrocarbons. In addition, it is also possible to use gelatin rectal capsules, which consist of a combination of the active compounds with a base. Possible base materials include, e.g., liquid triglycerides, polyethylene glycols, or paraffin hydrocarbons.

Suitable formulations for parenteral administration include aqueous solutions of the active compounds in water-soluble form, e.g., water-soluble salts and alkaline solutions. In addition, suspensions of the active compounds as appropriate oily injection suspensions may be administered. Suitable lipophilic solvents or vehicles include fatty oils or synthetic fatty acid esters. Aqueous injection suspensions may contain substances which increase the viscosity of the suspension include. Optionally, the suspension may also contain stabilizers.

In accordance with one aspect of the present invention, compounds of the invention are employed in topical and parenteral formulations and are used for the treatment of skin cancer.

The topical compositions of this invention are formulated preferably as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The preferred carriers are those in which the active ingredient is soluble. Emulsifiers, stabilizers, humectants and antioxidants may also be included, as well as agents imparting color or fragrance, if desired. Additionally, transdermal penetration enhancers may be employed in these topical formulations. Examples of such enhancers are found in U.S. Pat. Nos. 3,989,816 and 4,444,762.

Creams are preferably formulated from a mixture of mineral oil, self-emulsifying beeswax and water in which mixture of the active ingredient, dissolved in a small amount of an oil, such as almond oil, is admixed. A typical example of such a cream is one which includes approximately 40 parts water, approximately 20 parts beeswax, approximately 40 parts mineral oil and approximately 1 part almond oil.

Ointments may be formulated by mixing a solution of the active ingredient in a vegetable oil, such as almond oil, with warm soft paraffin and allowing the mixture to cool. A typical example of such an ointment is one which includes approximately 30% almond oil and approximately 70% white soft paraffin by weight.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### Example 1

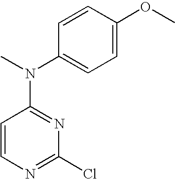

### (2-Chloro-pyrimidin-4-yl)-(4-methoxyphenyl)-methyl amine

A mixture of 2,4-dichloropyrimidine 0.447 g (3.0 mmol) and 4-methoxy-N-methylaniline 0.411 g (3.0 mmol) with conc. HCl aq 0.5 mL in iso-propanol 15 mL was stirred for 16 h at 23° C. Dichloromethane 30 mL was added to the reaction mixture, and the organic layer was neutralized with sat. Na₂CO₃ aqueous solution, water and dried over anhydrous MgSO₄. After removal of solvent, the crude product was purified with gradient column chromatography (EtOAc/hexanes, 0% to 50%) to give the titled compound in 90% yield. ¹H NMR (CDCl₃, 400 MHz): δ 7.84 (d, 1H, J=6.1 Hz), 7.14 (d, 2H, J=9.0 Hz), 6.98 (d, 2H, J=9.0 Hz), 6.09 (d, 1H, J=6.1 Hz), 3.85 (s, 3H), 3.45 (s, 3H). m/e: 250.4258 (M+1).

### Example 2

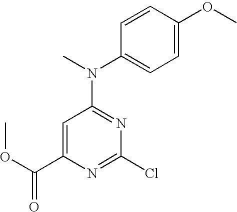

### Methyl 2-chloro-6-[(4-methoxyphenyl)(methyl)amino]pyrimidine-4-carboxylate

A mixture of methyl 2,4-dichloropyrimidine-6-carboxylate 0.566 g (2.0 mmol) and 4-methoxy-N-methylaniline 0.274 g (2.0 mmol) with conc. HCl aq 0.5 mL in iso-propanol 15 mL was stirred for 16 h at 23° C. Dichloromethane 30 mL was added to the reaction mixture, and the organic layer was neutralized with sat. Na₂CO₃ aqueous solution, water and dried over anhydrous MgSO₄. After removal of solvent, the crude product was purified with gradient column chromatography (EtOAc/hexanes, 0% to 50%) to give the titled compound in 70% yield. ¹H NMR (CDCl₃, 400 MHz): δ 7.14 (d, 2H, J=9.0 Hz), 7.00 (d, 2H, J=9.0 Hz), 6.84 (s, 1H), 3.90 (s, 3H), 3.87 (s, 3H), 3.49 (s, 3H). m/e: 308.1156 (M+1).

### Example 3

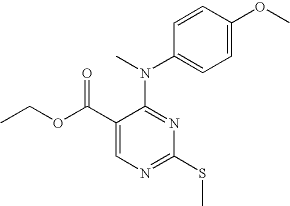

### Ethyl 4-[(4-methoxyphenyl)(methyl)amino]-2-(methylthio)pyrimidine-5-carboxylate

A mixture of ethyl 4-chloro-2-(methylthio)pyrimidine-5-carboxylate 0.466 g (2.0 mmol) and 4-methoxy-N-methylaniline 0.274 g (2.0 mmol) with conc. HCl aq 1 mL in iso-propanol 10 mL was stirred for 16 h at 23° C. Dichloromethane 30 mL was added to the reaction mixture, and the organic layer was neutralized with sat. Na₂CO₃ aqueous solution, water and dried over anhydrous MgSO₄. After removal of solvent, the crude product was purified with gradient column chromatography (EtOAc/hexanes, 0% to 50%) to give the titled compound in 75% yield. ¹H NMR (CDCl₃, 400 MHz): δ 8.26 (s, 1H), 7.07 (d, 2H, J=9.0 Hz), 6.87 (d, 2H, J=9.0 Hz), 3.80 (s, 3H), 3.68 (q, 2H, J=7.1 Hz), 3.53 (s, 3H), 2.57 (s, 3H), 1.07 (t, 3H, J=7.1 Hz). m/e: 334.1966 (M+1).

### Example 4

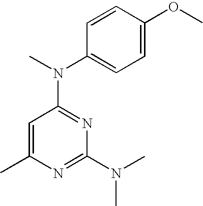

### N⁴-(4-Methoxyphenyl)-N²,N²,N⁴,6-tetramethylpyrimidine-2,4-diamine

The titled compound was obtained from a reaction of crude 2-chloro-N-(4-methoxyphenyl)-N,6-dimethylpyrimidin-4-amine and ethanolamine in DMF using microwave irradiation (120° C., 20 min). ¹H NMR (CD₃OD, 400 MHz): δ δ 7.23 (d, 2H, J=8.9 Hz), 7.07 (d, 2H, J=8.9 Hz), 5.59 (d, 1H, J=0.8 Hz), 3.85 (s, 3H), 3.51 (s, 3H), 3.29 (s, 6H), 2.21 (s, 3H). m/e: 273.2255 (M+1).

### Example 5

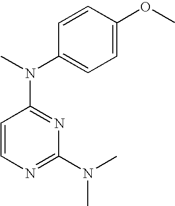

### N⁴-(4-Methoxyphenyl)-N²,N²,N⁴-trimethylpyrimidine-2,4-diamine

A mixture of 2-chloro-N-(4-methoxyphenyl)-N-methylpyrimidin-4-amine 0.125 g (0.5 mmol) and NH₄F 0.11 g (3.0 mmol) in DMF 2 mL was stirred at 120° C. for 24 h. Dichloromethane 20 mL was added to the reaction mixture, and the organic layer was washed with water, and dried over anhydrous MgSO₄. After removal of solvent, the crude product was purified with gradient column chromatography (EtOAc/hexanes, 0% to 70%) to give the titled compound in 92% yield. ¹H NMR (CDCl₃, 400 MHz): δ 7.77 (d, 1H, J=5.9 Hz), 7.14 (d, 2H, J=8.9 Hz), 6.93 (d, 2H, J=8.9 Hz), 5.51 (d, 1H, J=6.1 Hz), 3.83 (s, 3H), 3.41 (s, 3H), 3.17 (s, 6H). m/e: 259.4901 (M+1).

### Example 6

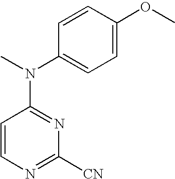

### 4-[(4-Methoxyphenyl)(methyl)amino]pyrimidine-2-carbonitrile

A mixture of 2-chloro-N-(4-methoxyphenyl)-N-methylpyrimidin-4-amine 0.250 g (1.0 mmol) and sodium cyanide 0.10 g (2.0 mmol) with DABCO 0.10 g (1.0 mmol) in DMSO 6 mL and iso-propanol 3 mL was stirred at 40° C. for 48 h. The reaction mixture was directly subjected to gradient column chromatography (EtOAc/hexanes, 0% to 70%) to give the titled compound in 82% yield. ¹H NMR (CDCl₃, 400 MHz): δ 8.01 (d, 1H, J=6.3 Hz), 7.14 (d, 2H, J=8.8 Hz), 7.00 (d, 2H, J=8.8 Hz), 6.30 (d, 1H, J=6.3 Hz), 3.86 (s, 3H), 3.46 (s, 3H). m/e: 241.1113 (M+1).

### Example 7

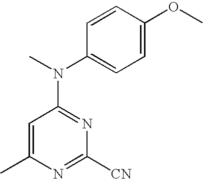

### 4-[(4-Methoxyphenyl)(methyl)amino]-6-methylpyrimidine-2-carbonitrile

A mixture of 2-chloro-N-(4-methoxyphenyl)-N,6-dimethylpyrimidin-4-amine 0.263 g (1.0 mmol) and sodium cyanide 0.10 g (2.0 mmol) with DABCO 0.10 g (1.0 mmol) in DMSO 6 mL and iso-propanol 3 mL was stirred at 40° C. for 48 h. The reaction mixture was directly subjected to gradient column chromatography (EtOAc/hexanes, 0% to 100%), followed by purification with preparative HPLC to give the titled compound in 70% yield as the corresponding mono-TFA salt. ¹H NMR (CDCl₃, 400 MHz): δ 7.14 (d, 2H, J=9.0 Hz), 7.01 (d, 2H, J=9.0 Hz), 6.14 (s, 1H), 3.87 (s, 3H), 3.45 (s, 3H), 2.52 (d, 3H, J=0.4 Hz). m/e: 255.1257 (M+1).

### Example 8

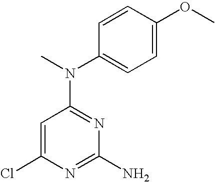

### 6-Chloro-N⁴-(4-methoxyphenyl)-N⁴-methylpyrimidine-2,4-diamine

A mixture of 2-amino-4,6-dichloropyrimidine 0.328 g (2.0 mmol) and 4-methoxy-N-methylaniline 0.274 g (2.0 mmol) with conc. HCl aq 1 mL in iso-propanol 10 mL was stirred for 24 h at 23° C. Dichloromethane 30 mL was added to the reaction mixture, and the organic layer was neutralized with sat. Na₂CO₃ aqueous solution, water and dried over anhydrous MgSO₄. After removal of solvent, the crude product was purified with gradient column chromatography (EtOAc/hexanes, 0% to 60%) to give the crude product, which was further purified with preparative HPLC to give the titled compound in 50% yield. ¹H NMR (CD₃OD, 400 MHz): δ 7.16 (d, 2H, J=9.0 Hz), 7.03 (d, 2H, J=9.0 Hz), 5.50 (s, 1H), 3.83 (s, 3H), 3.37 (s, 3H). m/e: 265.0859 (M+1).

### Example 9

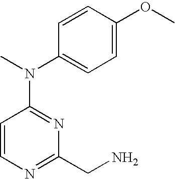

### 2-(Aminomethyl)-N-(4-methoxyphenyl)-N-methylpyrimidin-4-amine

A mixture of 4-[(4-methoxyphenyl)(methyl)amino]pyrimidine-2-carbonitrile 0.120 g (0.5 mmol) with conc. HCl 0.4 mL and 10% Pd/C 0.05 g in MeOH 10 mL was stirred at room temperature for 12 h under H₂ gas (1 atm). After filtration of Pd/C using Celite, the solution was concentrated, and the resulting solid was dissolved in CH₂Cl₂ 5 mL and 4M HCl in dioxane 1 mL. Removal of solvents gave a brown solid as a HCl salt in 98% yield calculated as a salt with HCl×1, which was shown to be >95% pure. ¹H NMR (CD₃OD, 400 MHz): δ 8.09 (d, 1H, J=7.4 Hz), 7.38 (d, 2H, J=8.8 Hz), 7.13 (d, 2H, J=8.8 Hz), 6.44 (d, 1H, J=7.4 Hz), 4.95 (br s, 2H), 3.87 (s, 3H), 3.74 (s, 3H). m/e: 245.2177 (M+1).

### Example 10

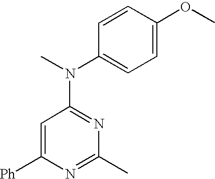

### 4-Methoxyphenyl)-methyl-(2-methyl-6-phenyl-pyrimidin-4-yl)amine

A mixture of ethyl benzylacetate 1.92 g (10.0 mmol) and acetamidine hydrochloride 5.0 g (53.0 mmol) with a ethanol solution (23% wt) of EtONa 20 mL (67.5 mmol) was stirred for 36 h under reflux. After removal of ethanol, the crude product was extracted with EtOAc, washed with water, and dried on anhydrous MgSO₄. After removal of solvents, the intermediate 2-methyl-6-phenylpyrimidin-4-ol was dried under reduced pressure, and used without further purification. A mixture of 2-methyl-6-phenylpyrimidin-4-ol 0.93 g (5.0 mmol) and POCl₃ 5 mL was heated under reflux for 3 h. After cooling, excess of POCl₃ was removed under reduced pressure, and the crude product was diluted with CHCl₃. The solution was poured into ice-water, washed with water, and dried on anhydrous MgSO₄. Removal of CHCl₃ gave 4-chloro-2-methyl-6-phenylpyrimidine (EMI-mass: 206 [M]⁺) in 90% yield (98% purity). A solution of 4-chloro-2-methyl-6-phenylpyrimidine 0.206 g (1.0 mmol) and 4-methoxy-N-methylaniline 0.137 g (1.0 mmol) with conc. HCl 0.2 mL in iso-propanol 5 mL was stirred at 23° C. for 16 h. The solution was concentrated under reduced pressure, diluted with CH₂Cl₂ 30 mL, washed with sat. Na₂CO₃ aqueous solution, and dried on anhydrous MgSO₄. After removal of solvents, the crude product was purified with gradient column chromatography (EtOAc/hexanes, 0% to 60%) to give titled product in 80% yield. ¹H NMR (CD₃OD, 400 MHz): δ 7.80-7.76 (m, 2H), 7.39-7.36 (m, 3H), 7.22 (d, 2H, J=9.0 Hz), 6.99 (d, 2H, J=9.0 Hz), 6.40 (s, 1H), 3.86 (s, 3H), 3.51 (s, 3H), 2.66 (s, 3H). m/e: 306.2506 (M+1).

### Example 11

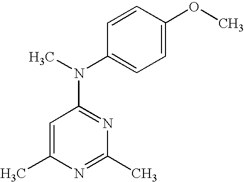

### (2,6-dimethyl-pyrimidin-4-yl)-(4-methoxyphenyl)-methylamine

To a stirring solution of 4-chloro-2,6-dimethyl-pyrimidine (7 mmol, 1 g), (4-methoxy-phenyl)-methyl-amine (7 mmol, 960 mg), and isopropanol (35 mL) was added concentrated HCl (4 mL). Stirred at room temperature overnight. Evaporated the isopropanol, added aq. Na₂CO₃, and extracted with dichloromethane. Dried the organic layer and concentrated. Chromatographed in 0 to 100% Ethyl Acetate in Hexanes to give 365 mg (21% yield). ¹H NMR (400 MHz) δ (ppm); 7.14 (d, J=9.0 Hz, 2H), 6.97 (d, J=9.0 Hz, 2H), 3.85 (s, 3H), 3.44 (s, 3H), 2.55 (s, 3H), 2.19 (s, Hz, 3H). LC/MS; 244.31 (M+1).

### Example 12

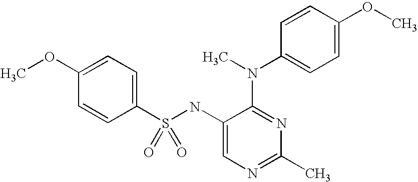

### 4-Methoxy-N-{4-[(4-methoxyphenyl)-methyl amino]-2-methyl-pyrimidin-5-yl)-benzenesulfonamide

N*4*-(4_Methoxy-phenyl)-2,N*4*-dimethyl-pyrimidine-4,5-diamine (76 mg, 0.31 mmol) was dissolved in 5 mL of pyridine. A solution of methoxybenzenesulfonyl chloride (64 mg, 0.31 mmol) was added in portion and the mixture was stirred at room temperature for 18 hours. The reaction mixture was concentrated and the resulting crud extract re-dissolved in ethyl acetate (30 mL), washed with water, dried above MgSO4, and concentrated. Purification was performed by preparative TLC (EtOAc/Hexane=1/1 as eluent) to give 36 mg of the title compound. 1H NMR (DMSO-d6) d 8.35-6.08 (m, 9H), 3.83 (s, 3H), 3.74 (s, 3H), 3.29 (s, 3H), 2.37 (s, 3H); 414 (M+H).

### Example 13

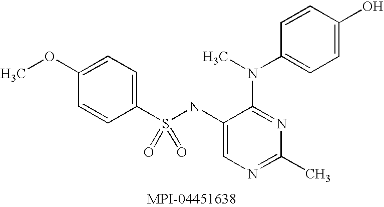

### N-{4-[(4-hydroxyphenyl)-methyl amino]-2-methyl-pyrimidin-5-yl)-4-methyoxy-benzenesulfonamide

The title compound was produced in the same manner as Example 12. ¹H NMR (DMSO-d) 7.45-6.73 (m, 9H), 3.82 (s, 3H), 3.25 (s, 3H), 2.45 (s, 3H); MS ESI⁺, 401(M+H).

### Example 14

### Identification of Cytotoxic Agents

A P388 murine leukemia cell line was obtained from NCI, Frederick, Md. P388 cells were cultured in RPMI-1640 supplemented with 10% fetal bovine serum, 2 mM Glutamax, 1 mM sodium pyruvate, 0.1 mM non-essential amino acids and 10 mM HEPES. Cells were grown at 37° C. in a humidified 5% CO₂ atmosphere. Exponentially growing P388 cells were plated at 5,000 cells/well in a 96-well flat-bottomed microtiter plate (Corning, Costar #3595). Twenty-four hours later, test compound was added to cells at final concentrations of 100 nM, 33.3 nM, 11.1 nM, 3.7 nM, 1.23 nM, 0.4 nM and 0.13 nM. Cellular viability was determined 72 hours later by measuring intracellular ATP with ATP-Lite assay system. The effect of compounds on cell viability was calculated by comparing the ATP levels of cells exposed to test compound with those of cells exposed to DMSO. A semi-log plot of relative ATP levels versus compound concentration was used to calculate the compound concentration required to inhibit growth by 50% (IC50). Data was analyzed by Prism software (GraphPad; San Diego, Calif.) by fitting it to a sigmoidal dose response curve.

The P388 IC50 data of representative compounds are summarized in Table I:

TABLE I
P388 IC50 Data

| Example Cmpd No. | P388 IC50 (nM) |
|---|---|
| 1 | 280 |
| 2 | 9000 |
| 3 | 54 |
| 4 | 1060 |
| 5 | 7900 |
| 6 | 6800 |
| 7 | 2500 |
| 8 | 1900 |
| 9 | 1900 |
| 10 | 1600 |
| 11 | 310 |
| 12 | 57 |
| 13 | 1400 |

Accordingly, compounds of the invention were identified as cytotoxic agents and are thus useful in treating the various diseases and disorders discussed above.

### Example 15

### Multidrug Resistant Cell Assays

Cytotoxicity of compounds in multidrug resistant cells can be determined by administering compounds to cell lines that overexpress the multidrug resistance pump MDR-1 and determining the viability of the cell lines. P388/ADR cell lines are known to overexpress the multidrug resistance pump MDR-1 (also known as P-glycoprotein-1; Pgp-1).

P388/ADR cell lines are obtained from American Type Culture Collection (Manassas, Va.) and maintained in RPMI-1640 media supplemented with 10% FCS, 10 units/ml penicillin and streptomycin, 2 mM Glutamax and 1 mM sodium pyruvate (Invitrogen Corporation, Carlsbad, Calif.). For compound testing, cells are plated in 96 well dishes at a concentration of 1.5×104 cells/well. Cells are allowed to adhere to the plate overnight and then incubated with compounds at final concentrations ranging from 0.13 nM to 10 uM for 72 hours. Cell viability is then assessed using the ATP-lite reagent (Perkin Elmer, Foster City, Calif.). Plates are read on a Wallac Topcount luminescence reader (Perkin Elmer, Foster City, Calif.) and the results graphed in Prism software (Graphpad Software, Inc., San Diego, Calif.). Non-linear regression with variable slope analysis is performed to obtain IC50 concentration values.

The P388/MDR IC50 data of representative compounds are summarized in Table II:

TABLE II
P388/MDR IC50 Data

| Example Cmpd No. | P388/MDR IC50 (nM) |
|---|---|
| 1 | 480 |
| 3 | 78 |
| 4 | 1100 |
| 5 | 6200 |
| 7 | 3900 |
| 8 | 2400 |
| 9 | 3100 |
| 10 | 800 |
| 11 | 400 |
| 12 | 62 |
| 13 | 7400 |

Accordingly, compounds of the invention were identified as cytotoxic agents in multidrug resistant cells and are thus useful in treating the various diseases and disorders discussed above in drug resistant cancer patients.

### Example 16

### Injection Formulation

| Excipients | Amount | |
|---|---|---|
| Active Compound | 5 | mg |
| PEG-400 | 5 | grams |
| TPGS | 10 | grams |
| Benzyl alcohol | 0.5 | gram |
| Ethanol | 2 | grams |

| | |
|---|---|
| D5W | Add to make 50 mL |

An injection formulation of a compound selected from Formula I (the “Active Compound”) can be prepared according to the following method. 5 mg of the Active Compound is dissolved into a mixture of the d-α-tocopheryl polyethylene glycol 1000 succinate (TPGS), PEG-400, ethanol, and benzyl alcohol. D5W is added to make a total volume of 50 mL and the solution is mixed. The resulting solution is filtered through a 0.2 μm disposable filter unit and is stored at 25° C. Solutions of varying strengths and volumes are prepared by altering the ratio of Active Compound in the mixture or changing the total amount of the solution.

### Example 17

### Tablet Formulation

| | | |
|---|---|---|
| Active Compound | 100.0 | mg |
| Lactose | 100.0 | mg |
| Corn Starch | 50.0 | mg |
| Hydrogenated Vegetable Oil | 10.0 | mg |
| Polyvinylpyrrolidone | 10.0 | mg |
| | 270.0 | mg |

A formulation of tablets of a compound selected from Formula I (the “Active Compound”) can be prepared according to the following method. 100 mg of Active Compound) is mixed with 100 mg lactose. A suitable amount of water for drying is added and the mixture is dried. The mixture is then blended with 50 mg of corn starch, 10 mg hydrogenated vegetable oil, and 10 mg polyvinylpyrrolidinone. The resulting granules are compressed into tablets. Tablets of varying strengths are prepared by altering the ratio of Active Compound in the mixture or changing the total weight of the tablet.

### Example 18

### Capsule Formulation

| | |
|---|---|
| Active Compound | 100.0 mg |
| Microcrystalline Cellulose | 200.0 mg |
| Corn Starch | 100.0 mg |
| Magnesium Stearate | 400.0 mg |
| | 800.0 mg |

A formulation of capsules containing 100.0 mg of a compound selected from Formula I (the “Active Compound”) can be prepared according to the following method. 100 mg of Active Compound is mixed with 200 mg of microcrystalline cellulose and 100 mg of corn starch. 400 mg of magnesium stearate is then blended into the mixture and the resulting blend is encapsulated into a gelatin capsule. Doses of varying strengths can be prepared by altering the ratio of the Active Compound to pharmaceutically acceptable carriers or changing the size of the capsule.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A compound according to Formula I: 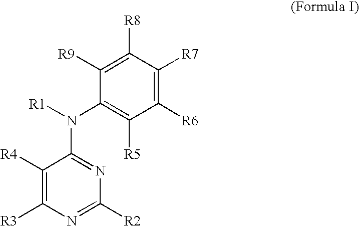
or a pharmaceutically acceptable salt thereof, wherein:
R₁ is methyl;
R₂ is C₁₋₆ alkyl, C₂₋₆ alkenyl, C₂₋₆ alkynyl, C₁₋₆ alkoxy, halo, C₁₋₆ haloalkyl, C₁₋₆ haloalkoxy, C₁₋₆ alkylthio, amino, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S(═O)₂NH₂, —S(═O)₂N(C₁₋₆ alkyl)₂, —S(═O)₂NH(C₁₋₆ alkyl), —S(═O)₂aryl, —S(═O)₂heteroaryl, —CN, —N₃, and —NO₂, each optionally substituted with one or more substituents;
R₃ and R₄ are independently H, C₁₋₆ alkyl, C₁₋₆ carbocyclic, C₁₋₆ alkylamino, C₂₋₆ alkenyl, C₂₋₆ alkynyl, C₁₋₆ alkoxy, halo, C₁₋₆ haloalkyl, C₁₋₆ haloalkoxy, C₁₋₆ alkylthio, amino, heterocyclic, aryl, heteroaryl, —N-aryl, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S(═O)₂NH₂, —S(═O)₂N(C₁₋₆ alkyl)₂, —S(═O)₂NH(C₁₋₆ alkyl), —S-aryl, —CN, —N₃, —NH₂ and —NO₂, each optionally substituted with one or more substituents;
R₅ and R₉ are independently H or F;
R₆ and R₈ are independently H, C₁₋₆ alkyl, C₁₋₆ carbocyclic, C₁₋₆ alkylamino, C₂₋₆ alkenyl, C₂₋₆ alkynyl, C₁₋₆ alkoxy, halo, C₁₋₆ haloalkyl, C₁₋₆ haloalkoxy, C₁₋₆ alkylthio, amino, heterocyclic, aryl, heteroaryl, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —N-aryl, —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S(═O)₂NH₂, —S(═O)₂N(C₁₋₆ alkyl)₂, —S(═O)₂NH(C₁₋₆ alkyl), —S-aryl, —S(═O)₂aryl, —S(═O)₂heteroaryl, —CN, —N₃, and —NO₂, each optionally substituted with one or more substituents;
R₇ is C₁₋₆ alkyl, C₁₋₆ carbocyclic, C₂₋₆ alkenyl, C₂₋₆ alkynyl, C₁₋₆ alkoxy, halo, halo-C₁₋₆ alkyl, halo-C₁₋₆ alkoxy, C₁₋₆ alkylthio, amino, heterocyclic, aryl, heteroaryl, —NH(C₁₋₆ alkoxy), —NH(C₁₋₆ alkyl)OH, —NHS(═O)₂(C₁₋₆ alkyl), —NHS(═O)₂(aryl), —NH(C₁₋₆ alkyl)C(═O)OH, —NH(C₁₋₆ alkyl)C(═O)O(C₁₋₆ alkyl), —N-aryl, —C(═O)OH, —C(═O)O(C₁₋₆ alkyl), —C(═O)NH₂, —C(═O)NH(C₁₋₆ alkyl), —C(═O)N(C₁₋₆ alkyl)₂, —OH, —O-aryl, —SH, C₁₋₆ haloalkylthio, —S-aryl, —S(═O)₂NH₂, —S(═O)₂N(C₁₋₆ alkyl)₂, —S(═O)₂NH(C₁₋₆ alkyl), —S(═O)₂-aryl, N—S(═O)₂NH₂—CN, —N₃, —NH₂ and —NO₂, each optionally substituted with one or more substituents;
with the provisos that:
1) when R₂ is NH₂ or NHCH₃, then R₃ is not methyl or substituted phenyl, R₄ is not NH₂ or halo;
2) when R₂ is halo, then R₃ is not methyl, R₄ is not halo, R₇ is not NH-acetate or N(Me)(Acetate);
3) when R₂ is methyl then R₃ is not N-aryl or R₄ is not NO₂; and
4) when R₂ is OH then R₃ is not OH.

2. The compound of claim 1 wherein R₂ is C₁₋₄ alkyl, C₂₋₄ alkenyl, C₂₋₄ alkynyl, C₁₋₄ alkoxy, halo, C₁₋₄ haloalkyl, C₁₋₄ haloalkoxy, C₁₋₄ alkylthio, amino, —NH(C₁₋₄ alkoxy), —NH(C₁₋₄ alkyl)OH, —NHS(═O)₂(C₁₋₄ alkyl), —NHS(═O)₂(aryl), —NH(C₁₋₄ alkyl)C(═O)OH, —NH(C₁₋₄ alkyl)C(═O)O(C₁₋₄ alkyl), —C(═O)OH, —C(═O)O(C₁₋₄ alkyl), —C(═O)NH₂, —C(═O)NH(C₁₋₄ alkyl), —C(═O)N(C₁₋₄ alkyl)₂, —OH, —O-aryl, —SH, C₁₋₄ haloalkylthio, —S(═O)₂NH₂, —S(═O)₂N(C₁₋₄ alkyl)₂, —S(═O)₂NH(C₁₋₄ alkyl), —CN, —N₃, and —NO₂, each optionally substituted with one or more substituents.

3. The compound of claim 1 wherein R₂ is methyl, CN, aminomethyl, Cl, SCH₃, NH₂, NHCH₃, NHCH₂CH₂OH, N(CH₃)₂, NH—OCH₃, or NHCH₂COOH.

4. The compound according to claim 1, wherein R₃ and R₄ are independently H, C₁₋₄ alkyl, C₁₋₄ carbocyclic, amino, C₁₋₄ alkoxy, halo, C₁₋₄ haloalkyl, C₁₋₄ haloalkoxy, heterocyclic, aryl, heteroaryl, —NHS(═O)₂(C₁₋₄ alkyl), —NHS(═O)₂(aryl), —NH(C₁₋₄ alkyl)C(═O)OH, —NH(C₁₋₄ alkyl)C(═O)O(C₁₋₄ alkyl), —C(═O)OH, —C(═O)O(C₁₋₄ alkyl), —C(═O)NH₂, —C(═O)NH(C₁₋₄ alkyl), —C(═O)N(C₁₋₄ alkyl)₂, —OH, —O-aryl, C₁₋₄ haloalkylthio, —S(═O)₂NH₂, —S(═O)₂N(C₁₋₄ alkyl)₂, —S(═O)₂NH(C₁₋₄ alkyl), —CN, —N₃, and —NO₂, each optionally substituted with one or more substituents.

5. The compound according to claim 1, wherein R₅, R₆, R₈ and R₉ are H or F.

6. The compound according to claim 1, wherein R₇ is C₁₋₄ alkyl, C₁₋₄ carbocyclic, C₂₋₄ alkenyl, C₂₋₄ alkynyl, C₁₋₄ alkoxy, halo, C₁₋₄ haloalkyl, C₁₋₄ haloalkoxy, C₁₋₄ alkylthio, amino, heterocyclic, aryl, heteroaryl, —NH(C₁₋₄ alkoxy), —NH(C₁₋₄ alkyl)OH, —NHS(═O)₂(C₁₋₄ alkyl), —NHS(═O)₂(aryl), —NH(C₁₋₄ alkyl)C(═O)OH, —NH(C₁₋₄ alkyl)C(═O)O(C₁₋₄ alkyl), —C(═O)OH, —C(═O)O(C₁₋₄ alkyl), —C(═O)NH₂, —C(═O)NH(C₁₋₄ alkyl), —C(═O)N(C₁₋₄ alkyl)₂, —OH, —O-aryl, —SH, C₁₋₄ haloalkylthio, —S(═O)₂NH₂, —S(═O)₂N(C₁₋₄ alkyl)₂, —S(═O)₂NH(C₁₋₄ alkyl), —CN, —N₃, —NH₂ and —NO₂, each optionally substituted with one or more substituents.

7. The compound according to claim 1, wherein R₇ is C₁₋₄ alkyl, —OH, C₁₋₄ alkoxy, —SH, C₁₋₄ alkylthio, —S(═O)₂NH₂, —S(═O)₂N(C₁₋₄ alkyl)₂, —S(═O)₂NH(C₁₋₄ alkyl), each optionally substituted with one or more substituents.

8. The compound according to claim 1, wherein:
R₁ is methyl;
R₂ is methyl, CN, aminomethyl, Cl, SCH₃, NH₂, NHCH₃, NHCH₂CH₂OH, N(CH₃)₂, NH—OCH₃, or NHCH₂COOH;
R₃ and R₄ are independently H, CH₃, COOH, COOCH₃, COOCH₂CH₃, phenyl, Cl or NHS(═O)₂(Ph-4-OCH₃);
R₅, R₆, R₈ and R₉ are H;
R₇ is OH or OCH₃;
with the provisos that:
1) when R₂ is NH₂ or NHCH₃, then R₃ is not methyl, R₄ is not NH₂ or halo;
2) when R₂ is halo, then R₃ is not methyl, R₄ is not halo; and
3) when R₂ is methyl then R₄ is not NO₂.

9. A compound selected from the group consisting of:
Ethyl 4-[(4-methoxyphenyl)(methyl)amino]-2-(methylthio)pyrimidine-5-carboxylate;
4-Methoxy-N-{4-[(4-methoxyphenyl)-methyl amino]-2-methyl-pyrimidin-5-yl)-benzenesulfonamide;
or a pharmaceutically acceptable salt thereof.

10. A compound selected from the group consisting of:
(2-Chloro-pyrimidin-4-yl)-(4-methoxyphenyl)-methyl amine;
(2,6-dimethyl-pyrimidin-4-yl)-(4-methoxyphenyl)-methylamine;
or a pharmaceutically acceptable salt thereof.

11. A compound selected from the group consisting of:
N⁴-(4-Methoxyphenyl)-N²,N²,N⁴,6-tetramethylpyrimidine-2,4-diamine;
4-[(4-Methoxyphenyl)(methyl)amino]-6-methylpyrimidine-2-carbonitrile;
6-Chloro-N⁴-(4-methoxyphenyl)-N⁴-methylpyrimidine-2,4-diamine;
2-(Aminomethyl)-N-(4-methoxyphenyl)-N-methylpyrimidin-4-amine;
4-Methoxyphenyl)-methyl-(2-methyl-6-phenyl-pyrimidin-4-yl)amine;
N-{4-[(4-hydroxyphenyl)-methylamino]-2-methyl-pyrimidin-5-yl)-4-methyoxy-benzenesulfonamide;
or a pharmaceutically acceptable salt thereof.

12. A compound selected from the group consisting of:
Methyl 2-chloro-6-[(4-methoxyphenyl)(methyl)amino]pyrimidine-4-carboxylate;
N⁴-(4-Methoxyphenyl)-N²,N²,N⁴-trimethylpyrimidine-2,4-diamine;
4-[(4-Methoxyphenyl)(methyl)amino]pyrimidine-2-carbonitrile;
or a pharmaceutically acceptable salt thereof.

13. A pharmaceutical composition comprising an effective amount of a compound according to claim 1 and a pharmaceutically acceptable carrier.

14. A pharmaceutical composition effective to inhibit neoplasia comprising a compound according to claim 1 and another anticancer agent selected from the group consisting of alkylating agents, antimitotic agents, topo I inhibitors, topo II inhibitors, RNA/DNA antimetabolites, DNA antimetabolites, EGFR inhibitors, proteosome inhibitors, antibodies, or a pharmaceutically acceptable salt or solvate of said agent.

15. A pharmaceutical composition effective to inhibit neoplasia comprising a bioconjugate of a compound according to claim 1 in bioconjugation with at least one known therapeutically useful antibody, growth factors, cytokines, or any molecule that binds to the cell surface.

16. A method of treating diseases that are responsive to cytotoxic agents, said method comprising treating a patient having a disease responsive to cytotoxic agents with a therapeutically effective amount of a compound according to claim 1.

17. The method of claim 16, wherein said compound is used in combination with another anticancer agent selected from alkylating agents, antimitotic agents, topo I inhibitors, topo II inhibitors, RNA/DNA antimetabolites, DNA antimetabolites, EGFR inhibitors, proteosome inhibitors, and antibodies, or a pharmaceutically acceptable salt of said another anticancer agent.

18. The method of claim 16, wherein said compound is used in combination with at least one agent selected from alpha-1-adrenoceptor antagonists, sigma-2 receptor agonists, HMG-CoA reductase inhibitors, HIV protease inhibitors, retinoid and synthetic retinoids, proteasome inhibitors, tyrosine kinase inhibitors, prenyl-protein transferase inhibitors, including farnesyl protein transferase inhibitors, inhibitors of geranylgeranyl-protein transferase type I (GGPTase-I) and geranylgeranyl-protein transferase type-II, cyclin-dependent kinase inhibitors, and COX-2 inhibitors, or a pharmaceutically acceptable salt of said agent.

19. A method of inhibiting neoplasia, said method comprising treating a patient having neoplasia with a therapeutically effective amount of a compound according to claim 1, wherein said compound is used in combination with radiation therapy.

20. A method for post-surgical treatment of cancer, said method comprising treating a patient in need of post-surgical treatment of cancer with a therapeutically effective amount of a compound according to claim 1.

21. A method of treating cancer, said method comprising treating a patient having cancer with a therapeutically effective amount of a compound according to claim 1.

22. The method of claim 21, wherein the patient to be treated is not responsive to another anticancer agent, or has developed resistance to such other anticancer agent.

23. The method of claim 21, wherein the patient to be treated is refractory to another anticancer agent.

24. The method of claim 22, wherein said other anticancer agent is selected from alkylating agents, antimitotic agents, topo I inhibitors, topo II inhibitors, RNA/DNA antimetabolites, EGFR inhibitors, angiogenesis inhibitors, tubulin inhibitors, proteosome inhibitors, alkylating agents, antimitotic agents, topo I inhibitors, topo II inhibitors, RNA/DNA antimetabolites, EGFR inhibitors, angiogenesis inhibitors, tubulin inhibitors, and proteosome inhibitors, or a pharmaceutically acceptable salt of said other anticancer agent.
